(19) United States
(12) Patent Application Publication
(10) Pub. No.: US 2010/0015201 A1
(43) Pub. Date: Jan. 21, 2010

(54) IMPLANT WITH COATING

(76) Inventors: Alexander Borck, Aurachtal (DE); Matthias Gratz, Erlangen (DE)

Correspondence Address: GREER, BURNS & CRAIN, 300 S WACKER DR, 25TH FLOOR, CHICAGO, IL, 60606, US

(21) Appl. No.: 12/505,184

(22) Filed: Jul. 17, 2009

(30) Foreign Application Priority Data
Jul. 21, 2008 (DE) 10 2008 040 572.8

(51) Int. Cl. A61F 2/00; A61K 31/519; A61P 7/00

(52) U.S. Cl. 424/423; 523/113; 514/262.1

(57) ABSTRACT

The present invention concerns an implant with a molecularly imprinted polymer coating, a process for its synthesis, a molecularly imprinted polymer, as well as a process for its synthesis and its use as well as a process for the treatment or prophylaxis of certain disease conditions by implantation of an implant in accordance with the invention according to the respective independent claims.

### FIELD

The present invention concerns an implant with a polymer coating, a process for its manufacture, a polymer as well as a process for its synthesis and its uses, and also a process for the treatment or prophylaxis of certain conditions of diseases by implantation of an implant according to the invention as per the respective independent claims.

### BACKGROUND

Implants are substances or parts that are inserted into a human or animal body to fulfill a certain replacement function for a limited period of time or for life. In contrast to transplants, implants consist of synthetic material (alloplasty). Often, a distinction is made between medical, plastic or functional implants.

Medical implants have the objective of supporting body functions or to replace such. Depending on the function, they are also described as implantable prosthesis. Known representatives are, for example, heart pace makers, brain pace makers for Parkinson's disease, heart implants, cochlear implants, implants in dental medicine, stents and implants that are intended to be a depot of a medication, as well as various forms of joint replacements.

Plastic implants are used in plastic surgery, for example, for the replacement of destroyed body parts or also for changing existing parts of the body.

Functional implants are designed for monitoring of human or animal functions by, for example, RFID (radio frequency identification) chips that are implanted under the skin.

By the numerous available implant types one can see that implants and their use in medicine have achieved great significance.

Because in classic treatment principles, as for example in the systemic application of one or several active ingredients, sometimes significant side effects are to be expected, such as, for example, in the treatment of tumors, a local, controlled release of active ingredient at the target location or in its proximity is gaining increasing significance (local drug delivery concept; LDD concept). In order to be able to implement this local application of active ingredients, especially basic implant bodies are coated with one or more active ingredients that are implanted either at the target location or in its proximity in a human or animal organism and thus release active ingredients locally. This clinically established method is used millions of times per year worldwide and it is to be expected that upon consideration of a demoscopic adjustment within the age pyramid, the need for new active ingredients as well as new methods of administration will be growing.

In the orthopedic area, infections and thromboembolic complications are known in connection with endoprosthetic implants. Thromboemboly is an acute venous or arterial blood vessel block which can be caused by a displaced thrombus which can be created by thrombocytes that adhere to the surface of the implant.

In the area of heart circulation diseases, minimally invasive forms of therapy offer a steadily expanding further treatment therapy for expanding and stabilizing narrowed coronary vessels by percutaneous transluminal coronary angioplasty (PTCA) and stent implants. As a late complication, in addition to the renewed narrowing of the vessel according to PTCA (so-called in-stent-restenosis, (ISR) and vessel inflammation, primarily the risk of a thrombosis embolism is to be mentioned. Given this background, especially stents of prior art are made available that are coated with active ingredients that are intended to counteract one or more of the sequelae.

The implants that release active ingredients of prior art are accordingly coated with one or more active ingredients of specified concentration and they release such, if appropriate, according to a defined release regimen in the physiological surroundings of a human or animal body. These implants that are coated with active ingredient of prior art do, however, not offer a flexible release of active ingredients in a situation (occurrence of a sequela) that is coordinated as needed or the blocking of physiological reactions as an answer to the implantation of the implant, for example, inadequate proliferation of smooth muscle cells, excess formation and secretion of components of the extracellular matrix, etc.

The objective of the present invention is to make an implant available which has one or more of the following characteristics given the physiological conditions in a human or animal organism:
binding with i) one or more physiological compounds of a human or animal organism, especially
   enhancing growth factors, enzymes for modification/reduction of extracellular matrix, enhancing enzymes for dissolving fibrin for the enhancement of enzymes for activating systemically administered prodrugs,
   blocking receptors for growth factors or blocking of receptors of cell activation and/or
binding with ii) one or more systemically administered active ingredients.

### SUMMARY OF THE INVENTION

The present problem is partially or completely solved by the substance matters of the independent claims.

Accordingly, a first subject matter of the invention concerns an implant for implantation in a human or animal organism with a polymer coating, characterized by, that the polymer coating is molecularly imprinted and, subject to the physiological conditions in a human or animal organism the molecular imprint is suitable to bind with

i) one or more physiological compounds of a human or animal organism and/or

ii) one or more systemically administered active ingredients.

A second subject matter of the invention concerns a process for manufacturing a polymer-coated implant in accordance with the invention.

A third subject matter of the invention concerns a molecularly imprinted polymer that is suitable as coating for an implant according to the invention, characterized by, that the molecular imprint, subject to the physiological conditions in a human or animal organism, is suitable to bind with

i) one or more physiological compounds of a human or animal organism and/or

ii) one or more systemically administered active ingredients.

A fourth subject matter of the invention concerns a process for the synthesis of a molecularly imprinted polymer in accordance with the invention, characterized by, that the process comprises the following steps:

a) Preparation of
i) one or more physiological compounds of a human or animal organism and/or
ii) one or more systemically administered active ingredients,

b) Preparation of one or more materials that can be polymerized,

c) Preparation of one or more polymerization initiators,

d) Mixing of i) the compound or compounds of ii) the active ingredient or the active ingredients from step a) with the materials that can be polymerized from step b) as well as with the polymerization initiator or polymerization initiators from step c),

e) Polymerizing the mixture from step d) and

f) Removing i) the compound or compounds and/or ii) the active ingredient or active ingredients at least from the surface of the polymer by washing the polymer of step e) with one or more suitable solvents and/or mixtures of solvents.

A fifth subject matter of the present invention concerns a use of a molecularly imprinted polymer in accordance with the invention as coating for an implant according to the invention.

A sixth subject matter of the invention concerns an application of the molecularly imprinted polymer for the manufacture of the implant according to the invention.

A seventh subject matter of the invention concerns a process for the treatment or prophylaxis of a stenosis and the treatment of diseases of the blood vessels or of tissue that is supplied by these blood vessels, including tumors, characterized by, that an implant in accordance with the invention is implanted into a human or animal organism.

Preferred embodiments of the subjects in accordance with the invention are shown in the dependent claims as well as in the following detailed description and are, to the extent this makes sense, combinable among one another.

### BRIEF DESCRIPTION OF THE DRAWINGS

FIG. 1 shows a schematic process for the synthesis of a molecularly imprinted polymer according to the invention.

### DETAILED DESCRIPTION OF THE INVENTION

The invention is based on the surprising insight that as a result of an implant in accordance with the invention with a molecularly imprinted polymer coating or a molecularly imprinted polymer in accordance with the invention, according to the respective independent claims i) binds one or more physiological compounds of a human or animal organism and/or ii) one or more active ingredients subject to the physiological conditions in a human or animal organism are bound to the surface of the polymer and thus by utilizing mechanisms that are natural in the body
growth factors for the improvement of endothelialization, especially in the blood vessel system in stenosis, enzymes for the modification/reduction of extra cellular matrix, enzymes for dissolving fibrin and/or enzymes for splitting systematically administered prodrugs are enhanced,
receptors for growth factors for proliferation control or receptors for cell activation for manipulation of immune cells are blocked and/or
systemically administered active ingredients are bound to the polymer surface,
making possible a subsequent, and thus a loading as needed of active ingredient of the implant and thereby also the release of the active ingredient suitable to need (for example, necessary when other clinical disease patterns are present, particularly diabetes mellitus),
making possible that an active ingredient concentration is obtained over a predefined period of time and thus repeated applications,
making possible a change of active ingredients of, for example, accentuated proliferation-blocking (e.g. paclitaxel) to accentuated endothelial promoting or antithrombosis acting,
making possible a parallel local vasodilation therapy,
making possible the influencing of the effects of a local pH value adjustment, especially in the degradation of degradable metal stents, preferably magnesium stents.

Implants or basic implant bodies within the meaning of the present invention can represent all medical, plastic and/or functional implants or basic implant bodies and are, for example, selected from a group consisting of heart pace makers, brain pace makers; heart implants; pace maker electrodes; defibrillator electrodes; cochlear implants; implants for dentistry, endoprosthesis, preferably for knee joints and hip joints; depot implants that are designed to form a depot of an active ingredient; degradable or permanent, coronary or peripheral stents; degradable or permanent stents for other cavities, preferably the esophagus, the bile duct, the urinary tract, the prostate or the trachea; and local drug delivery (LDD) implants, that are preferably implanted endovascularly in the blood or in other cavities.

In a preferred embodiment of the present invention, implants in accordance with the invention are selected from a group consisting of heart pace makers; heart implants; pace maker electrodes; defibrillator electrodes; degradable or permanent coronary or peripheral stents; and local drug delivery (LDD) implants that are preferably implanted endovascularly in blood or in other cavities.

In a particularly preferred embodiment of the present invention, implants in accordance with the invention are selected from the group of permanent or degradable coronary stents (coronary stents), whereby the material of the basic body of the stent can include metal and/or polymers.

The original mechanical functions of a coronary stent, for example, the ability to dilate, the small recoil, the stability over a desired period of time (in the case of degradable stents, for example, consisting of magnesium and its alloys) as well as the flexibility should preferably be present in stents in accordance with the invention as implants in accordance to the invention.

In the following, implant materials that are to be used in accordance with the invention, preferably basic stent body materials, as well as preferred embodiments of such are described.

Degradable Basic Implant Body, Preferably, Degradable Basic Stent Body:

Within the meaning of the present invention, “degradable (basic) implant (body)” means preferred “degradable stent (basic body)” that a basic body degrades in physiological surroundings, particularly in the vessel system of a human or animal organism, i.e. it is degraded in such a way that the stent loses its integrity. Preferably, basic degradable bodies in accordance with the invention degrade only when the function of the implant no longer makes physiological sense or is no longer necessary. For degradable stents this means that the stent preferably degrades only then when it loses its integrity, when the traumatized tissue of the vessel is healed and the stent thus must no longer remain in the lumen of the vessel.

### Metallic Basic Body:

In a preferred embodiment in accordance with the invention, the degradable material includes or consists preferably of a metallic material, which is a bio-corrodible alloy, whereby the main component of the alloy is selected from the group of magnesium, iron, zinc and/or wolfram; especially preferred for a degradable metallic material is a magnesium alloy.

The alloy, particularly comprising magnesium, iron, zinc and/or wolfram is to be selected in its composition in such a way that it is bio-corrodible. Within the meaning of the present invention, those alloys are described as bio-corrodible for which degradation takes place in a physiological environment that leads to, in the end, that the entire stent or the part of the stent that is made of the working material loses its mechanical integrity. In the present case, a metallic micro-structure is understood to be an alloy whose primary component is magnesium, iron, zinc and/or wolfram. The main component is that component of the alloy that has the highest proportion of weight in the alloy. Preferably, the proportion of the primary component amounts to more than 50 percent by weight, more preferred, more than 70 percent by weight. Of these, a magnesium alloy is particularly preferred.

If the material in accordance with the invention is a magnesium alloy, it preferably contains yttrium and other rare earth metals, as an alloy of this type distinguishes itself because of its physico-chemical characteristics and its high biocompatibility, particularly also its degradation products.

Particularly preferred is the use of the yttrium (W) and rare earth (E) containing biocorrodible magnesium alloys (WE43 & WE54 of magnesium electron) with a proportion of magnesium of >90 percent by weight, yttrium 3.7-5.5 percent by weight, rare earth metals 1.5-4.4 percent by weight and the remainder <1 percent by weight for the manufacture of implants (stents) that is described in EP 1 419 793 B1.

In experiments and in first clinical tests, these magnesium alloys have already confirmed their special suitability, i.e. they show high biocompatibility, favorable processing characteristics, good mechanical properties and an adequate corrosion behavior for the application purposes.

Under the collective term “Rare Earths” RE the following are to be understood in the present application scandium (21) and yttrium (39) as well as the “Light Rare Earths” LRE lanthan (57), cerium (58), neodymium (60), and promethium (61) and the “Heavy Rare Earths” HRE samarium (62), europium (63), gadolinium (64), terbium (65), dysprosium (66), holmium (67), erbium (68), thulium (69), ytterbium (70) and lutetium (71).

### Basic Polymer Body:

Basic implant bodies in accordance with the invention, preferably basic stent bodies can comprise—according to a further alternative embodiment according to the invention—de-gradable polymers or consist of such, preferably selected from the group consisting of polydioxanone; polyglycolide; polycaprolactone; polyhydroxyvalerianic acid; Polyhydroxybutyric acid; Polylactide, preferably poly(L-lactide), poly(D-lactide), poly(D,L-lactide) and blends, as well as copolymers, and preferably poly(L-lactide-co-glycolide), poly(D,L-lactide-co-glycolide), poly(L-lactide-co-D-L-lactide), poly(L-lactide-co-trimethylenecarbonate) and tri-blockcopolymers; polysaccharide, preferably chitosan, levan, hyaluronic acid, heparin, dextran and cellulose.

### Permanent Basic Implant Body, Preferably Permanent Basic Stent Body:

In contrast to degradable basic bodies, the “permanent basic implant body” preferably the “permanent basic stent body” is not essentially degraded in the physiological environment in the human or animal organism, so that it does not lose its integrity.

In a further preferred embodiment according to the invention, the basic body comprises or consists of a permanent implant, preferably a permanent stent of a shape memory material, preferably of one or more materials selected from the group of nickel-titanium alloys and copper-zinc-aluminum alloys, more preferred nitinol.

In a particularly preferred embodiment in accordance with the invention, the basic body comprises or consists of a permanent implant, preferably a permanent stent made of stainless steel, preferably of Cr—Ni—Fe steel, here preferably the alloy 316L, or a Co—Cr steel.

In a further preferred embodiment, the basic implant body, preferably basic stent body can additionally comprise plastic, preferably polyetherurethane, and/or ceramic and/or additional polymer coatings.

If, within the meaning of the present invention, endovascularly implantable stents, preferably coronary stents are used as implantable basic bodies, this way, all usual stent geometries can be used for the present invention. Particularly preferred are stent geometries that are described especially in U.S. Pat. No. 6,896,695, US 2006/241742, U.S. Pat. No. 5,968,083 (Tenax), EP 1 430 854 (Helix-Design), U.S. Pat. No. 6,197,047 and EP 0 884 985.

The basic implant body in accordance with the invention, preferably the basic stent body including the above preferred embodiments is provided with a polymer coating that is molecularly imprinted in accordance with the invention. The polymer for the polymer coating in accordance with the invention is preferably selected from the group consisting of co-polymerisates of one or more acrylates and/or methacrylates with acrylacetate (has hydrophilic groups that can easily be saponified to OH groups) acrylamine, Acrylic acids, vinylpyrrolidone, styrol (interaction with aromatic epitopes), vinylpyridine or vinylsaccharideco-polmerisates. Vinylsaccharides can be synthesized as per the following literature: http://www.digibib.tu-bs.de/?docid=0000124; ISBN 3-89825-567-0, Anke Wiegand and http://bib11p1.rz.tu-bs.de/docportal/content/below/index.xml. The selection of monomers is strongly dependent on the template for which the polymer is to be supplied with binding affinity. Should a polymer with selectivity for an aromatic compound or naphthalene be synthesized, the use of styrol makes sense. For molecules that carry amino groups, monomers that have an acid group or an OH group are suitable. The described vinylsaccharides can interact with a large number of templates.

According to a preferred embodiment in accordance with the invention, the molecular characteristic of the implant according to the invention, preferably stent is suitable to bind—subject to the physiological conditions in a human or animal organism i) one or more physiological compounds that is or that are selected from the group consisting of growth factors, preferably transforming growth factor, fibroblast growth factor angiopoietin 2 and/or vascular endothelial growth factor, enzymes for the modification or reduction of extracellular matrix, preferably matrix-metalloproteinases and thereby particularly preferred matrix-metalloproteinase1, enzymes for dissolving fibrin such as, but not exclusively, plasmin, but preferred its activators or in combination with such, thereby preferred tissue plasminogen activator and/or urokinase plasminogen activator, enzymes for activation of systemically administered prodrugs, for example, esterases, enzymes from the series of hydrolases, particularly lipases and proteases, enzymes from the series of the isomerases, receptors for growth factors, preferred, but not limited to receptors for the platelet-derived growth factor.

Within the meaning of the present invention, “active ingredient” is a substance or a compound that is not physiologically present in a human or an animal organism and that causes a biologic reaction in a human or animal organism.

In a preferred embodiment of the implant according to the invention, preferably the stent in accordance with the invention, the polymer coating is molecularly imprinted to that effect that it ii) binds one or more systematically administered active ingredients, which make control of the formation of extracellular matrix or intimal proliferation possible, i.e. act proliferation-blocking, for example, paclitaxel or rapamycine and its derivates, act endothelially promoting, for example, statines, such as, for example, atorvastatin or cerivastatin, acting antithrombogenic and/or anticoagulating, for example, dipyridamol, heparin, comarin derivatives, for example, marcumar, acetylsalicylic acid and/or act clopidogrel, vasodilating, for example, dipyridamol, NO or nitrogenmonoxide donators, such as, for example isosorbiddinitrate, nitroprusside or molsidomine, calcium antagonists, ACE-blocker such as captopril, angiotensin-II-receptor-subtyp-1 antagonists, such as, for example, losartane, potassium channel opener, such as, minoxidil or diazoxide and/or endothelial-receptor antagonists such as, for example, bosentane or sitaxentane, and/or that can counteract the biological effect of a local pH value adjustment into the basic range by degradable metal stents, preferably magnesium stents. Biological effects that occur in the basic range in a local pH value adjustment after the implantation of metal stents, preferably magnesium stents, as well as active ingredients that counteract these biological effects are described in DE 10 2006 038 235 A1 and are to that extent incorporated herein by reference to DE 10 2006 038 235 A1.

In a further preferred embodiment, the polymer coating of the implant in accordance with the invention comprises, preferably the stent in accordance with the invention, additionally one or more active ingredients, preferably selected from the group consisting of active ingredients which make control of the formation of extracellular matrix or intimation proliferation possible, i.e. that act proliferation-blocking, for example, paclitaxel or rapamycin and its derivatives, act endothelially promoting, for example, statines, such as, for example atorvastatin or cerivastatin, act antithrombogenic and/or anticoagulating, for example, dipyridamol, heparin, comarin derivatives, for example, marcumar, acetylsalicylic acid and/or act clopidogrel, vasodilating, for example, dipyridamol, NO or nitrogenmonoxide donators, such as, for example, isosorbiddinitrate, nitroprusside or molsidomine, ACE-blocker such as captopril, angiotensin-II-receptor-subtyp-1 antagonists, such as, for example, losartane, potassium channel openers such as minoxidil or diazoxide and/or endothelial receptor antagonists, such as, for example, bosentane or sitaxentane, and/or can counteract the biological effect of a local pH value adjustment into the basic range by degradable metal stents, preferably magnesium stents. Biological effects that occur in a local pH value adjustment into the basic range after the implantation of metal stents, preferably magnesium stents, as well as active ingredients that counteract these biological effects are described in DE 10 2006 038 235 A1 and are to that extent incorporated herein by reference to DE 102006038235 A1.

### Example of an Embodiment

Synthesis of a polymer matrix with binding selectivity for dipyridamol Composition of polymers: 60% methacrylic acid, 30% acrylic acid or acrylate or acrylate derivative, 10% vinylpyrrolidon in acetone, alternatively, THF and ethanol are suitable, whereby the concentration of active ingredient is limited in these solvents.

After dissolving 10 g of active ingredient (0.5-10 g) in 100 ml acetone, 6 g methacrylic acid, 3 g acrylic acid and 1 g vinylpyrrolidone are added.

The mixture is stirred and reacted with 2 mg AIBN and it is polymerized under a UV lamp (230 nm). In the first hour of polymerization, the reaction is vigorously swirled every 15 minutes (10 sec.).

The solid substance that is created is removed after 6 hours, pounded and washed with 100 ml water.

The release of dipyridamol from the polymer matrix can be determined with HPLC analytics.

Coating of the Basic Stent Body with Molecularly Imprinted and Loaded Polymer:

Optionally, the basic stent body is sprayed with methyl-2-cyanoacrylate in anhydrous conditions. The coating with methyl-2-cyanoacrylate is particularly suitable as adhesion layer for the subsequently applied molecularly imprinted polymer. The moist stent is subsequently transported into a dust chamber in which the pulverized and washed polymer with molecular recognition for the template (dipyridamol) is located. After precipitation of the polymer with molecular imprint as dust on the stent, the stent is removed from the dust chamber and exposed to humidity of at least 60%.

The reaction is shown in the following:

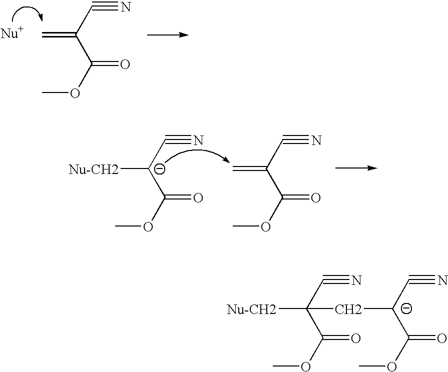

Here, water acts as nucleophile and starts the anionic polymerization. The polymer is thus affixed to the surface of the implant.

Additional preferred compounds that can be applied as adhesion layer onto the stent surface are selected from the group consisting of derivatives of cyanoacrylate and of diisocyanate. Particularly preferred derivates of diisocyanate are hexamethylenediisocyanate (HMDI), toluoldiisocynanate (TDI), diisocyanateodicyclohexylmethane (H12MDI) and isophorondiisocyanate (IPDI). The coating of the surface of the stent with an adhesive agent is then particularly advantageous in order to apply increased quantities of the pulverized molecularly imprinted polymer on the surface of the stent.

Preferred embodiments of the polymer materials or i) the one or more physiological compounds of a human or animal organism and/or ii) the one or more systemically administered active ingredients of the first substance matter of the invention can also be preferred embodiments for the third subject matter in accordance with the invention, namely the molecularly imprinted polymer according to the invention itself.

According to the fourth subject matter of the invention, as per step a) i) one or more physiological compounds of a human or animal organism and/or ii) one or more active ingredients that can be administered systemically are prepared. Preferred embodiments is hereto, that have already been described in the first subject matter of the invention can also be applied to the fourth subject matter of the invention.

According to step b) of the synthesis according to the invention for the molecularly imprinted polymer, one or more materials that can be polymerized are prepared. Preferred embodiments hereof were also already described in the first subject matter according to the invention and the monomers for this are also applicable to the fourth subject matter of the invention.

According to step c) of the synthesis of a molecularly imprinted polymer in accordance with the invention, one or more polymerization initiators are prepared. Within the meaning of the present invention, polymerization initiators are usually all substances or compounds which make a polymerization possible of the materials that can be polymerized that were prepared in step b) of the process according to the invention. Preferred is or are the polymerization initiators selected from the group consisting of 2,2-aco-bis-isobutyronitril (AIBN). According to step d) of the synthesis according to the invention of a molecularly imprinted polymer the i) the compound or compounds or ii) the active ingredient or the active ingredients from step a) are mixed with the materials that can be polymerized from step b) as well as the polymerization initiator or polymerization initiatiors from step c). This can for one be done in substance but also in a suitable solvent or solvent mixture. In a preferred embodiment, the compound or the compounds from step a) are first dissolved in step d) in a solvent or a solvent mixture. For this, all typically suitable solvents or solvent mixtures can be selected that can also be mixed with the materials that can be polymerized from step b). Preferred is the solvent or solvent mixture that is selected from the group consisting of acetone, dioxane, chloroform, tetrahydrofuran and/or water.

As a solvent, acetone is particularly preferred as in contrast to ethanol and tetrahydrofuran no, or only a small limitation is to be expected with respect to the concentration of the compounds to be dissolved from step a).

According to step e) of the synthesis in accordance with the invention for a molecularly imprinted polymer, the mixture from step d) is polymerized according to customary procedures. In a preferred embodiment, the polymerization in step e) is effected by UV irradiation.

According to step f) of the synthesis in accordance with the invention for a molecularly imprinted polymer i) the compound or the compounds or ii) the active ingredient or the active ingredients are removed by washing the polymer at least from the surface of the polymer from step e) with one or more suitable solvents or mixtures or solvents, so that the molecularly imprinted polymer is suitable under physiological conditions i) to bind anew the physiological compound or the physiological compounds and/or ii) the systemically administered active ingredient or active ingredients. For this, usually all suitable solvents and/or solvent mixtures can be selected that lead to the removal of i) the compound or compounds or ii) of the active ingredient or active ingredients. In a preferred embodiment, the suitable solvent or the suitable solvents and/or solvent mixtures are selected from the group consisting of methanol, ethanol and/or water.

Preferably, the washing step f) is repeated two, three or several times. In a particularly preferred embodiment, first washing takes place in a solvent mixture of methanol: ethanol at a volume relationship in the range of 4:1 to 1:4, further preferred of 1:1 is used and if necessary, subsequently washed with water.

According to the fifth subject matter of the invention, the use of a molecularly imprinted polymer according to the invention is claimed as coating for an implant in accordance with the invention. The preferred embodiments for the implant in accordance with the invention as per the first subject matter of the invention and/or the molecularly imprinted polymer according to the third subject matter of the invention as well as the synthesis of a molecularly imprinted polymer as per the fourth subject matter of the invention are also applicable to the fifth subject matter of the invention.

The sixth subject matter of the invention concerns use of the molecularly imprinted polymer according to the invention for the manufacture of a polymer-coated implant according to the invention. Here too, the preferred embodiments of an implant in accordance with the invention as per the first subject matter of the invention of a molecularly imprinted polymer according to the invention as per the third subject matter of the invention as well as the manufacturing process for an implant according to the invention or a molecularly imprinted polymer as per the second and fourth subject matter of the invention are applicable to the sixth subject matter of the invention.

Finally, according to the seventh subject matter of the invention a process for the treatment or prophylaxis of a stenosis or for the treatment of diseases of the blood vessels or of tissue that is supplied by these vessels, including tumors it is claimed which is characterized thereby, that an implant according to the invention is implanted into a human or animal organism. Here too, the preferred embodiments for the implant according to the invention as per the first subject matter of the invention, if necessary by consulting the manufacturing process for the implant according to the invention as per the second subject matter according to the invention and if necessary the molecularly imprinted polymer according to the invention as per the third subject matter of the invention as well as its synthesis as per the fourth subject matter of the invention are also applicable to the preferred embodiments for the seventh subject matter of the invention.

### DESCRIPTION OF FIGURE

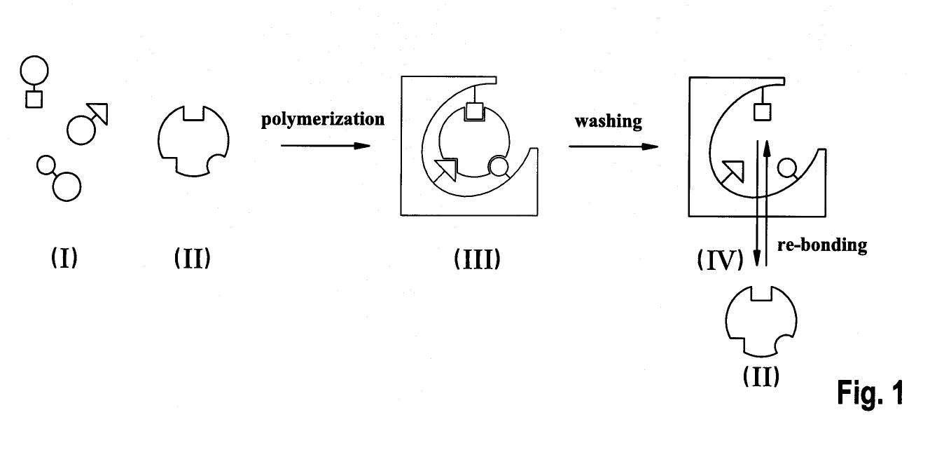

FIG. 1 shows a schematic illustration of a synthesis process according to the invention for a polymer that is molecularly imprinted in accordance with the invention.

In FIG. 1 (I) represents the materials that can be polymerized according to step b) of the synthesis according to the invention as per the fourth subject matter of the invention.

(II) i) represents the physiological compound or compounds of a human or animal organism and/or ii) the systemically administrated active ingredients as per step a) (the so-called template or templates) of the synthesis for a molecularly imprinted polymer in accordance with the invention as per the fourth subject matter of the invention.

After polymerization, the materials that can be polymerized (I) are present with the template or templates II as compound (III). The bonds between (I) and (II) can be covalent, ionic, hydrogen bonds or other bonds. Usually, such compounds are selected that can be reversibly dissolved because of the washing processes in order to make a bonding of i) of the physiological compound (s) and/or ii) the systematically administered active ingredient (s) possible.

Accordingly, the polymer (IV) that is imprinted according to the invention comprises one or more, preferably selective binding sites for the template or templates (II), also i) the physiological compound or physiological compounds that react in a human or animal organism and/or ii) the systematically administered active ingredient or active ingredients that, subject to physiological compounds, then again form a bond with the imprinted polymer according to the invention.

The preferred embodiments of the third or fourth subject matter of the invention are also applicable to the description of the figures.

### Examples of Embodiments

In the following, the present invention is described in examples of embodiments that do, however, not limit the scope of protection of the claimed subject matters in accordance with the invention as per the independent claims.

### Example of Embodiment 1: Synthesis of a Polymer Molecularly Imprinted with Dipyridamol According to the Invention

10 g Dipyridamol active ingredient, approx. 100 ml acetone, 6 g methacrylic acid, 3 g acrylic acid and 1 g vinylpyrrolidon and 2 mg 2,2-azobisisobutyronitril (AIBN) are prepared (steps a) to c) using the synthesis according to the invention for the molecularly imprinted polymer according to the invention).

First, the 10 g of the active ingredient dipyridamol are dissolved in 100 ml acetone and subsequently, this mixture is mixed with 6 g methacrylic acid, 3 g acrylic acid and 1 g vinylpyrrolidon as well as with 2 mg 2,2-azobisisobutyronitril (AIBN) while stirring (step d) of the synthesis according to the invention for the molecularly imprinted polymer according to the invention).

The mixture continues to be stirred and polymerized under a UV lamp (230 nm) for 6 hours, whereby within the first hour of polymerization, the mixture is vigorously swirled every 15 minutes (10 sec.) (step e) of the synthesis according to the invention of the molecularly imprinted polymer in accordance with the invention).

The polymer that is obtained as solid matter is pounded after 6 hours and washed with a 100 ml solvent mixture of ethanol/methanol (50:50 by percent of volume). Subsequently, the polymer is washed with 100 ml water.

The binding of the molecularly imprinted polymer according to the invention with the active ingredient dipyridamol can be determined analytically with high pressure liquid chromatography (HPLC) by using decreasing concentrations of a physiological parent solution of dipyridamol.

### Example of Embodiment 2: Synthesis of a Molecularly Imprinted Polymer in Accordance with the Invention

Synthesis of a polymer matrix with binding selectivity for dipyridamol Composition of polymers 60% methacrylic acid, 30% acrylic acid, 10% vinylpyrrolidon in acetone, alternatively THF and ethanol are suitable, whereby the concentration of active ingredient is limited in these solvents.

After dissolving 10 g of active ingredient (0.5-10 g) in 100 ml acetone, 6 g methacrylic acid, 3 g acrylic acid and 1 g vinylpyrrolidon are added.

The mixture is stirred and reacted with 2 mg AIBN and polymerized under a UV lamp (230 nm). During the first hour of polymerization, the reaction is vigorously swirled every 15 minutes (10 sec.).

The solid substance that is obtained is removed after 6 hours, pounded and washed with 100 ml ethanol/methanol (50:50). Subsequently, it is washed with 100 ml of water.

The selectivity for dipyridamol is analytically determined by HPLC by using decreasing concentrations of a parent solution.

A polymer is present that has binding affinities for dipyridamol.

### Example of Embodiment 3: Manufacture of a Coated Implant According to the Invention

Coating of a basic stent body with an adhesive layer and a molecularly imprinted polymer.

The basic stent body is sprayed with methyl-2-cyanoacrylate in anhydrous conditions. The moist stent is then placed into a dust chamber in which the pulverized and washed polymer with molecular recognition for the template (dipyridamol) is located. After precipitation of the molecularly imprinted polymer as dust on the stent, the stent is removed from the dust chamber and exposed to humidity of at least 60%.

### Example of Embodiment 4: Synthesis of a Polymer Molecularly Imprinted with Dipridamol According to the Invention

10 g of the active ingredient dipyridamol, approx. 100 ml acetone, 6 g methacrylic acid, 3 g methacrylate and 1 g vinylpyrrolidon and 2 mg 2,2-azobisisobutyronitril (AIBN) are prepared (step a) to c) in the synthesis process according to the invention for the molecularly imprinted polymer according to the invention).

First, the active ingredient of 10 g dipyridamol is dissolved in 100 ml acetone and subsequently, this mixture is mixed with 6 g methacrylic acid, 3 g methacrylate and 1 g vinylpyrrolidon as well as with 2 mg 2,2-azobisisobutyronitril (AIBN) while stirring (step d) of the synthesis according to the invention for the molecularly imprinted polymer according to the invention).

The mixture continues to be stirred and polymerized under a UV lamp (230 nm) for 6 hours, whereby during the first hour of polymerization, the mixture is vigorously swirled every 15 minutes (10 sec.) (step e) of the synthesis according to the invention for the molecularly imprinted polymer according to the invention).

The polymer that is obtained is pounded as solid substance after 6 hours and washed with 100 ml of a solvent mixture of ethanol/methanol (50:50 by percent of volume). Subsequently, the polymer is washed with 100 ml water. After drying, one obtains a pulverized molecularly imprinted polymer with binding selectivity for dipyridamol.

The binding of the molecularly imprinted polymer in accordance with the invention with the active ingredient dipyridamol can be determined analytically with high pressure liquid chromatography (HPLC) by using decreasing concentrations of a physiological parent solution of dipyridamol.

### Example of Embodiment 5: Synthesis of a Polymer Molecularly Imprinted with Dipyridamol According to the Invention

10 g of the active ingredient dipyridamol, approx. 100 ml acetone, 6 g methacrylic acid, 3 g ethylacrylate and 1 g vinylpyrrolidon and 2 mg 2,2-azobisisobutyronitril (AIBN) are prepared (step a) to c) in the synthesis process according to the invention of the molecularly imprinted polymer in accordance with the invention).

First, the 10 g of the active ingredient dipyridamol are dissolved in 100 ml acetone and subsequently, this mixture is mixed with 6 g methacrylic acid, 3 g ethylacrylate and 1 g vinylpyrrolidon as well as with 2 mg 2,2-azobisisobutyronitril (AIBN) while stirring (step d) of the synthesis in accordance with the invention for the molecularly imprinted polymer according to the invention).

The mixture continues to be stirred and polymerized under a UV lamp (230 nm) for 6 hours, whereby within the first hour of the polymerization the mixture is vigorously swirled every 15 minutes (10 sec.) (step e) of the synthesis process according to the invention for the molecularly imprinted polymer according to the invention).

The polymer that is obtained as solid matter is pounded after 6 hours and washed with 100 ml of a solvent mixture of ethanol/methanol (50:50 by percent volume). Subsequently, the polymer is washed with 100 ml water. After drying, one obtains a pulverized, molecularly imprinted polymer with binding selectivity for dipyridamol.

The binding of the molecularly imprinted polymer with the active ingredient dipyridamol according to the invention can be analytically determined by high pressure liquid chromatography (HPLC) by using decreasing concentrations of a physiological parent solution of dipyridamol.

The reaction is shown in the following:

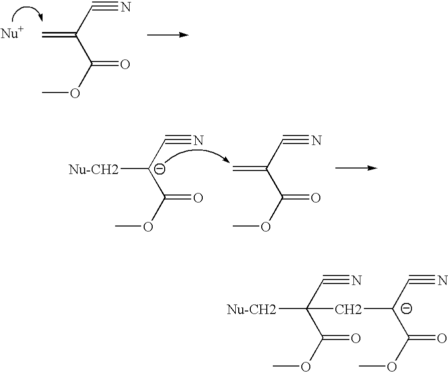

Here, water acts as a nucleophile and starts the anionic polymerization. In this way, the polymer is bonded to the surface of the implant.

1. An implant for implantation in a human or animal organism with a polymer coating characterized by, that the polymer coating is molecularly imprinted and the molecular imprint is suitable for binding with, under the physiological conditions in a human or animal organism, at least one of:
i) one or more physiological compounds of a human or an animal organism and,
ii) one or more systemically administered active ingredients.

2. An implant according to claim 1, characterized by, that the polymer is selected from a group consisting of copolymerisates of one or more acrylates and/or methacrylates with one or more of acrylacetate, acrylamine, acrylic acid, vinylpyrrolidon, styrol, vinylpyridine and vinylsaccharide-copolymerisates.

3. An implant according to claim 1, characterized by, that
i) the one or more physiological compounds are selected from the group consisting of growth factors, one or more enzymes for the modification or reduction of extracellular matrix, one or more enzymes for dissolving fibrin, one or more enzymes for activating systemically administered prodrugs, one or more enzymes from the series of hydrolases, enzymes from the series of isomerases, and one or more receptors for growth factors.

4. An implant according to claim 1, characterized by, that
ii) the one or more active ingredients are selected from a group consisting of cell proliferation blockers, cytostatic drugs, immunosuppressive drugs, statins, antithrombogens and active ingredients with anticoagulating effect, vasodilatators potassium channel openers, nitrogenmonoxide donators, ACE-blockers, angiotensin-II-receptor-subtyp-1-antagonists,
endothelial-receptor antagonists and active ingredients that can counteract a pH value adjustment into the basic range.

5. An implant according to claim 1, characterized by, that the implant is selected from the group consisting of heart pace makers; brain pacemakers; coronary implants; pace maker electrodes; defibrillation electrodes; cochlear implants; dental implants; endoprosthesis; depot implants that are designed to build a depot of an active ingredient; degradable or permanent coronary or peripheral stents; degradable or permanent stents for other cavities; and local drug delivery (LLD) implants.

6. A process for the manufacture of a polymer-coated implant according to claim 1, characterized by, that the molecularly imprinted polymer is affixed to the surface of the implant and thereby, that the molecularly imprinted polymer comes in contact with body fluids.

7. A molecularly imprinted polymer suitable as coating for a polymer-coated implant according to claim 1, characterized by, that the molecular imprint is suitable to bind with, under the physiological conditions of a human or animal organism, at least one of:
i) one or more physiological compounds of a human or an animal organism and
ii) one or more systemically administered active ingredients.

8. A synthesis process for obtaining a molecularly imprinted polymer according to claim 7, characterized by, that the synthesis process comprises the following steps:
a) preparation of at least one of:
i) one or more physiological compounds of a human or an animal organism and
ii) one or more systemically administered active ingredients,
b) preparation of one or more materials that can be polymerized,
c) preparation of one or more polymerization initiators,
d) mixing the one or more physiological compounds or the one or more systemically administered active ingredients from step a) with the one or more materials that can be polymerized from step b) as well as with the one or more polymerization initiators from step c),
e) polymerization of the mixture from step d) to make a polymer, and,
f) removal of the one or more physiological compounds or the one or more systemically administered active ingredients at least from the surface of the polymer obtained from step e) by washing the polymer with one or more suitable solvents or solvent mixtures.

9. A process according to claim 8, characterized by, that the polymerization initiator from step c) is 2,2-azobisisobutyronitril (AIBN).

10. A process according to claim 8, characterized by, that in step d) the one or more physiological compounds from step a) are first dissolved in a solution containing one or more materials selected from the group consisting of acetone, dioxane, water, chloroform, tetrahydrofuran and water.

11. A process according to claim 8, characterized by, that in step e) polymerization takes place by UV irradiation.

12. A process according to claim 8, characterized by, that in step f) the suitable solvent ardor solvent mixtures are selected from the group consisting of methanol, ethanol and water.

13. A molecularly imprinted polymer according to claim 7 configured as a coating for a polymer-coated implant for a human or an animal organism.

14. A molecularly imprinted polymer according to claim 7 configured for manufacturing a polymer-coated implant according to claim 1.

15. An implant according to claim 1 configured for one or more of the treatment or prophylaxis of a stenosis, for treatment of diseases of the blood vessels, and of diseases of tissues supplied by blood vessels, including tumors.

16. An implant according to claim 3 wherein:
the one or more growth factors is one or more of a transforming growth factor, fibroblast growth factor, angiopoietin 2 and a vascular endothelial growth factor; and,
the one or more enzymes for the modification or reduction of extracellular matrix is one or more of matrix-metalloproteinases and matrix-metalloproteinase 1.

17. An implant according to claim 3 wherein:
the one or more enzymes for dissolving fibrin is one or more of plasmin, plasmin activators, tissue plasminogenactivator, and urokinase plasminogenactivator; and,
the one or more enzymes for activating systemically administered prodrugs is one or more esterases;

18. An implant according to claim 3 wherein:
the one or more enzymes from the series of hydrolases is one or more of lipases and proteases; and,
the one or more receptors for growth factors is one or more receptors for the platelet-derived growth factor.

19. An implant according to claim 3 wherein:
the one or more growth factors is one or more of a transforming growth factor, fibroblast growth factor, angiopoietin 2 and a vascular endothelial growth factor;
the one or more enzymes for the modification or reduction of extracellular matrix is one or more of matrix-metalloproteinases and matrix-metalloproteinase 1;
the one or more enzymes for dissolving fibrin is one or more of plasmin, plasmin activators, tissue plasminogenactivator, and urokinase plasminogenactivator;
the one or more enzymes for activating systemically administered prodrugs is one or more esterases;
the one or more enzymes from the series of hydrolases is one or more of lipases and proteases; and,
the one or more receptors for growth factors is one or more receptors for the platelet-derived growth factor.

20. An implant according to claim 4 wherein the vasodilatators are one or more of dipyridamol or calciumantagonists.
